(19) United States
(12) Patent Application Publication
(10) Pub. No.: US 2014/0148686 A1
(43) Pub. Date: May 29, 2014

(54) SUPINE BREAST MRI

(71) Applicant: Gilbert Thevathasan, Brampton (CA)

(72) Inventor: Gilbert Thevathasan, Brampton (CA)

(21) Appl. No.: 14/007,803

(22) PCT Filed: Jun. 8, 2012
(86) PCT No.: PCT/IB2012/002089

§ 371 Date: Dec. 26, 2013

Related U.S. Application Data
(60) Provisional application No. 61/495,620, filed on Jun. 10, 2011.

(51) Int. Cl. A61B 5/00; A61B 5/055

(52) U.S. Cl. CPC A61B 5/708; A61B 5/0555; A61B 5/4312
USPC 600/415

(57) ABSTRACT

Systems and methods for imaging tissue of a patient positioned in a supine position is disclosed. The system includes an imaging array comprising a flexible mounting element, wherein the flexible mounting element includes at least a first articulating member configured to flex into position responsive to pressure and hold a flexed position upon release of pressure. In another example, the imaging array includes a curved portion, sized and dimensioned for positioning adjacent to a naturally positioned breast in response to articulation of the at least one articulating member of the flexible mounting element. In another example, the imaging array includes an RF coil array disposed on the flexible mounting element.

### RELATED APPLICATIONS

This application claims priority under 35 U.S.C. §119(e) to U.S. Provisional Application Ser. No. 61/495,620, entitled “SUPINE BREAST MRI,” filed on Jun. 10, 2011, which application is incorporated by reference herein in its entirety.

### BACKGROUND

Breast cancer is one of the leading causes of death in women. As a result, early detection is important, and screening of all women is encouraged. Efficient and effective methods for screening are therefore needed in the health care industry.

Diagnosis and treatment by noninvasive MRI is one method that has been shown to greatly enhance both detection, and survival of breast cancer. In conventional breast MRI, a local “breast” RF coil is utilized to receive signals from breast tissues being examined. Conventional breast coils, however, are typically designed to image the breast when the patient is in a prone position. Further breast coils are typically designed to accommodate a large volume of interest corresponding to a statistically large breast size to provide a uniform signal to noise ratio over the large image area. Other custom coils have been developed to address breast size issues. These conventional coils, include custom coils for different sized breast volume are configured to capture MRI images with the patient in a prone position. In some conventional systems, prone MRI images are then translated into images of how the breast should appear once the patient is positioned for surgery, that is, in a supine position.

### SUMMARY

As conventional MRI imaging systems are configured to image breast tissue in the prone position, such conventional approaches require complex translation mechanisms to present to medical personnel images of how the breast will appear, for example, during surgery. During staging for medical procedures including, for example, lumpectomy (removal of breast tissue) the patient is positioned on an operating table in a supine position. MRI imaging taken of the patient in a prone position can fail to provide adequate guidance for patients who are staged in the supine position for surgery. For example, prone imaging can fail to adequately define where cancerous material is located within the breast when the patient is in a supine position, and further can fail to provide adequate guidance on where borders should be drawn to minimize the volume of tissue removed.

Therefore, aspects and embodiments are directed to systems and methods that are configured to accurately obtain an MRI image of the breast and/or surrounding tissue while a patient is in a supine position. Accordingly there is provided systems and methods for supine MRI imaging of breasts, that can incorporate positionable supine breast coils. The positionable supine breast coils can include articulating members that enable contoured positioning of the supine coils adjacent to a naturally positioned breast (i.e., the position the breast takes when in supine position). Contoured positioning enables the supine breast coil to track approximately the contour of the breast being imaged while maintaining some minimal spacing between the coil and the imaged tissue, and in some embodiments can further account for the variety of size, shape, contour, and volume of breast tissue in patient populations.

According to one aspect an imaging array for imaging tissue of a patient positioned in a supine position is provided. The imaging array comprises a flexible mounting element, wherein the flexible mounting element includes at least one articulating member configured to flex into position responsive to pressure and to hold a flexed position upon release of pressure and a curved portion, sized and dimensioned for positioning adjacent to a naturally positioned breast in response to articulation of the at least one articulating member of the flexible mounting element and an RF coil array disposed on the flexible mounting element.

According to one embodiment, the imaging array further comprises a positioning assembly configured to position the flexible mounting element adjacent to the supine positioned patient. According to one embodiment, the positioning assembly is further configured to position the flexible mounting element adjacent to the supine positioned patient without deforming the naturally positioned breast while the supine positioned patient is breathing. According to one embodiment, the positioning assembly comprises components selected from a group including articulating components, telescoping components and flexible components. According to one embodiment, the at least one articulating member is constructed and arranged of a plurality of articulating connectors.

According to one embodiment, each of the plurality of articulating connectors are configured to hold a fixed position until a threshold pressure is applied, and wherein the plurality of articulating connectors are configured to permit movement when the threshold pressure is exceeded. According to one embodiment, the plurality of articulating connectors include pressure fit joints.

According to one embodiment, the flexible mounting element includes a configuration having a generally flat flexible portion adjacent to the curved portion. According to one embodiment, the generally flat flexible portion the flexible mounting element is configured to conform to contours of a body of the supine positioned patient adjacent the breast and positioned to permit breathing without touching the body of the supine positioned patient. According to one embodiment, curved portion is configured to conform to a contour of a plurality of breast sizes responsive to articulation of the at least one articulating member of the flexible mounting element. According to one embodiment, the curved portion is positionable for imaging the naturally positioned breast during breathing without touching any imaged tissue.

According to another aspect, a method of obtaining an Magnetic Resonance Image (MRI) of an object is provided. The method includes the steps of positioning an RF coil for use in MRI imaging above the breast of a patient that is in a supine imaging position on a transportable patient bed, contouring the RF coil to conform to the shape of the patient's breast, moving the transportable patient bed into an MRI scanner and obtaining an MRI image, and while the patient remains in the supine imaging position, providing medical treatment to the patient, the medical treatment guided by at least some information in the MRI image. According to one embodiment, the method further comprises an act of maintaining spacing between the RF coil and the patient's body. According to one embodiment, the method further comprises preserving the spacing during breathing by the patient. According to one embodiment, contouring the RF coil includes applying a threshold pressure to the RF coil permitting shaping of the RF coil to conform the shape of the patient's breast.

According to another aspect, a method of obtaining an Magnetic Resonance Image (MRI) of an object is provided. The method includes the steps of positioning an RF coil for use in MRI imaging above the breast of a patient that is in a supine imaging position on a transportable patient bed, contouring the RF coil to conform to the shape of the patient's breast, moving the transportable patient bed into an MRI scanner and obtaining an MRI image. According to one embodiment, the method further comprises an act of maintaining spacing between the RF coil and the patient's body. According to one embodiment, the method further comprises preserving the spacing during breathing by the patient. According to one embodiment, contouring the RF coil includes applying a threshold pressure to the RF coil permitting shaping of the RF coil to conform the shape of the patient's breast.

According to one aspect, an imaging array for imaging tissue of a patient positioned in a supine position is provided. The imaging array comprises a flexible mounting element, wherein the flexible mounting element includes at least a first articulating member configured to flex into position responsive to pressure and hold a flexed position upon release of pressure, an RF coil array disposed on the flexible mounting element, and a cup-shaped region formed on the flexible mounting element, wherein the cup-shaped region is configured to be sized and dimensioned responsive to articulation of the at least one articulating member of the flexible mounting element, and wherein the cup-shaped region is further configured to be positioned adjacent to a naturally positioned breast while facing the supine positioned patient.

According to one embodiment, the imaging array further comprises a positioning assembly configured to position the flexible mounting element adjacent to the supine positioned patient. According to one embodiment, the positioning assembly is further configured to position the flexible mounting element adjacent to the supine positioned patient without deforming the naturally positioned breast while the supine positioned patient is breathing. According to one embodiment, the positioning assembly includes a positioning arm. According to one embodiment, the positioning assembly further comprises a hanger arm extending from a distal end of the positioning arm and connected to the flexible mounting element. According to one embodiment, the hanger arm includes at least one articulating connector configured to position the flexible mounting element relative to the positioning arm.

According to one embodiment, the at least the first articulating member is constructed and arranged of a plurality of articulating connectors. According to one embodiment, each of the plurality of articulating connectors are configured to hold a fixed position until a threshold pressure is applied, and wherein the plurality of articulating connectors are configured to permit movement when the threshold pressure is exceeded. According to one embodiment, the plurality of articulating connectors include pressure fit joints. According to one embodiment, the plurality of articulating connectors include ball-and-socket joints.

According to one embodiment, the flexible mounting element includes a configuration having a generally flat flexible portion adjacent to the cup-shaped region. According to one embodiment, the generally flat flexible portion the flexible mounting element is configured to conform to contours of a body of the supine positioned patient adjacent the breast and positioned to permit breathing without touching the body of the supine positioned patient.

According to one embodiment, the cup-shaped region is configured to conform to a contour of a plurality of breast sizes responsive to articulation of the at least the first articulating member of the flexible mounting element. According to one embodiment, the cup-shaped region is configured to preserve spacing between the cup-shaped region and the naturally positioned breast during breathing. According to one embodiment, the cup-shaped region is positioned for imaging the naturally positioned breast during breathing without touching any imaged tissue.

According to one aspect, an imaging array for imaging tissue of a patient positioned in a supine position is provided. The imaging array comprises a flexible mounting element that includes at least a first articulating member having a first position and that is configured to flex into a flexed position responsive to pressure and hold the flexed position upon release of pressure, an RF coil array disposed on the flexible mounting element, and a cup-shaped region formed on the flexible mounting element, wherein the cup-shaped region is configured to be sized and dimensioned responsive to articulation of the at least one articulating member of the flexible mounting element.

According to one embodiment, the imaging array further comprises a positioning assembly configured to position the flexible mounting element adjacent to the supine positioned patient. According to one embodiment, the positioning assembly is further configured to provide for positioning the imaging array adjacent to the naturally positioned breast of the supine positioned patient. According to one embodiment, the positioning assembly includes a positioning arm. According to one embodiment, the positioning assembly further comprises a hanger arm extending from a distal end of the positioning arm and connected to the flexible mounting element. According to one embodiment, the hanger arm includes at least one articulating connector configured to position the flexible mounting element relative to the positioning arm.

According to one embodiment, the at least the first articulating member is constructed and arranged of a plurality of articulating connectors. According to one embodiment, each of the plurality of articulating connectors are configured to hold the first position until a threshold pressure is applied, and wherein the plurality of articulating connectors are configured to permit movement into the flexed position when the threshold pressure is exceeded. According to one embodiment, the plurality of articulating connectors include pressure fit joints. According to one embodiment, the flexible mounting element includes a flat flexible portion adjacent to the cup-shaped region. According to one embodiment, the flat flexible portion of the flexible mounting element is configured to conform to contours of a body of the supine positioned patient.

According to one embodiment, the cup-shaped region is configured to conform to a contour of a plurality of breast sizes responsive to articulation of the at least the first articulating member of the flexible mounting element. According to one embodiment, the cup-shaped region is sized and arranged so that it can be positioned adjacent the naturally positioned breast in the supine position of the patient without touching any imaged tissue. According to one embodiment, the cup-shaped region is configured to preserve spacing between the cup-shaped region and the naturally positioned breast during breathing.

Still other aspects, embodiments, and advantages of these exemplary aspects and embodiments, are discussed in detail below. Any embodiment disclosed herein may be combined with any other embodiment in any manner consistent with at least one of the objects, aims, and needs disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. The appearances of such terms herein are not necessarily all referring to the same embodiment. The accompanying drawings are included to provide illustration and a further understanding of the various aspects and embodiments, and are incorporated in and constitute a part of this specification. The drawings, together with the remainder of the specification, serve to explain principles and operations of the described and claimed aspects and embodiments.

### BRIEF DESCRIPTION OF THE DRAWINGS

Various aspects of at least one embodiment are discussed below with reference to the accompanying figures, which are not intended to be drawn to scale. Where technical features in the figures, detailed description or any claim are followed by references signs, the reference signs have been included for the sole purpose of increasing the intelligibility of the figures, detailed description, and claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. The figures are provided for the purposes of illustration and explanation and are not intended as a definition of the limits of the invention. In the figures:

FIG. 1 is a perspective view of a patient positioned on a patient support adjacent a MRI system;

FIG. 2 illustrates a coil support structure including a supine coil assembly, according to one embodiment of the present invention;

FIGS. 3A-B illustrate example supine coil assemblies, according to embodiments of the present invention; and

FIG. 4 illustrates an exploded view of a supine RF coil array, according to one embodiment of the present invention.

### DETAILED DESCRIPTION

According to aspects of this disclosure, various structures and methods are provided herein for imaging a breast of a patient positioned in a supine position, while allowing for the patient to be positioned in the supine position and to account for a patients breathing. In at least one embodiment, various structures and methods are provided for conforming an imaging array to a size and shape of a patient breast, and for imaging a patient breast while the patient is in the supine position. One advantage of imaging the patient's breast in the supine position is that the image corresponds readily to how the patient's breast will be viewed by medical personnel during an operations.

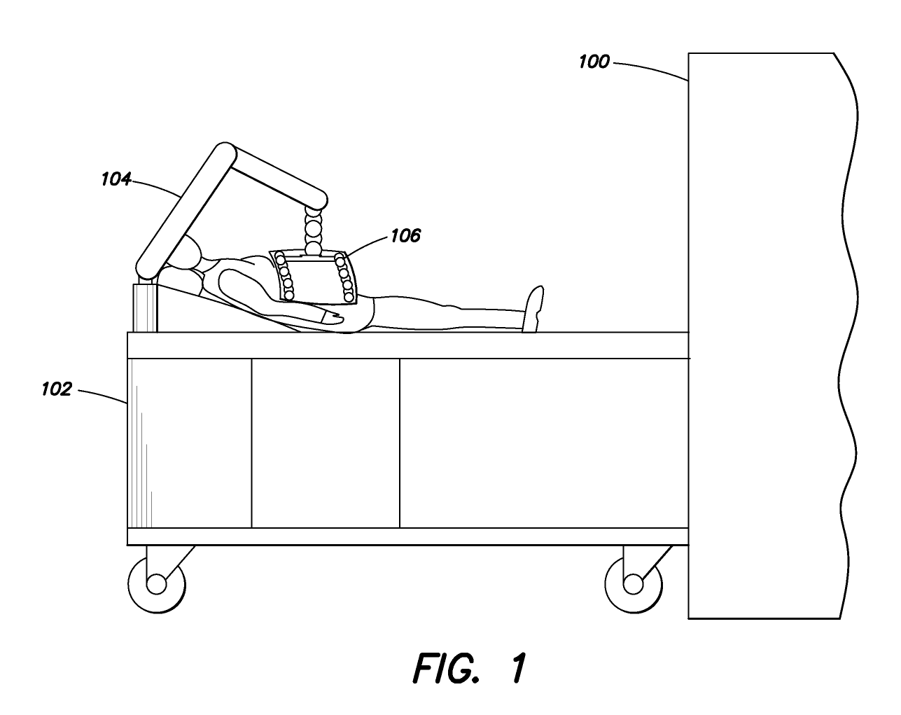

By way of introduction, referring now to the figures and more particularly to FIG. 1, a patient is shown on a patient transport 102 having a coil support structure 104. The coil support structure 104 can be positioned on the patient transport 102 adjacent an MRI scanner 100. The patient can then be moved into the bore of the MRI scanner for imaging. The coil support structure 104, as shown here, allows the patient to lie face up, i.e., in a supine position, with the breasts positioned adjacent to a supine RF coil array 106 for imaging, as described more thoroughly below.

According to some aspects, MRI imaging of patients in a supine position enables capture of high quality unilateral supine breast images. High quality supine breast imaging can be used to facilitate image-aid of clinical procedures (e.g., lumpectomy, mastectomy, etc.) which are typically performed by medical personnel when the patient is in a supine position. In one embodiment, an imaging coil and support structure may be used in conjunction with a whole body 1.5T MR scanner (e.g., 106) which can include an number of available models and/or brand of scanner (e.g., GE Signa Excite). According to one embodiment, high quality diagnostic images of the patients breast and surrounding tissue may be obtained using a unilateral four-element receive coil for supine breast MR imaging. The receive coil elements can have a variety of configurations, discussed in greater detail below. In one example, the elements are configured to have a size of 26×23 cm. In the example elements, the four-element receive coil may be constructed an arranged of 6.35 mm wide copper tape. Further, in some embodiments, coupling between coil loops was minimized by overlapping neighboring loops. Further embodiments also include architectures to decouple capacitors, and still further embodiments also include low-impedance pre-amplifiers, which features can be implemented to improve signal-to-noise (“SNR”) ratios for image capture.

According to some embodiments, the coil support structure (e.g. 104) can be constructed and arranged to support the supine coil above the breast of the patient on a standard bed of a scanner or a separate patient transport. FIG. 1 illustrates one example architecture of a coil support structure, however, in other embodiments many different types of coil support structures, including flexible arms, telescoping arms, articulated arms, and various combinations thereof may be used; accordingly the present invention is not limited to the use of any particular type of coil support structure. Rather, the coil support structure can include any architecture that is configured to place a supine coil proximate to a patient for imaging. In some embodiments, the support structure can be lifted and/or moved to allow convenient patient positioning on a patient bed. Once the patient is in position, the coil support structure is moved into a position above the supine patient. A supine RF coil array 106 can be attached to the coil support structure via connection that enable further positioning of the supine RF coil array 106. In some embodiments, the supine coil array can be positioned above the breast based on positioning of the coil support structured and then repositioned using, for example, a connection mechanism (e.g., gimbal joint, permitting manipulation in multiple directions). In one example, the coil support structure can include a gimbaled joint or other articulating structure (e.g., Loc-Line system, Modular Hose, etc.) which connects the supine coil array to the support structure, enabling any direction of rotation and/or tilting of the supine coil above the patient's breast. The positioning may be controlled manually, automatically via a computer controlled retraction/extension mechanism, or via some combination thereof.

As discussed briefly above, the supine coil itself may also be modified to obtain a position and/or shape above the patient's breast so that the supine coil can conform as closely as possible to different breast geometries. The degree of spacing between the coil and the patients breast may be, but need not be, uniform. In some embodiments, positioning and/or shaping of the supine coil array is manually performed to insure that no contact with the patient's breast occurs or such that contact is minimal, thereby to minimize deformation of the breast by the coil array. Further, in some implementations spacing between the supine coil array and the patient can be maintained to insure that even during breathing, no contact occurs. In one example, an air gap having a minimal width of approximately 1 cm is maintained between the positioned coil array and the breast skin. In some examples, sufficient air gaps can be visually confirmed by medical personnel during images.

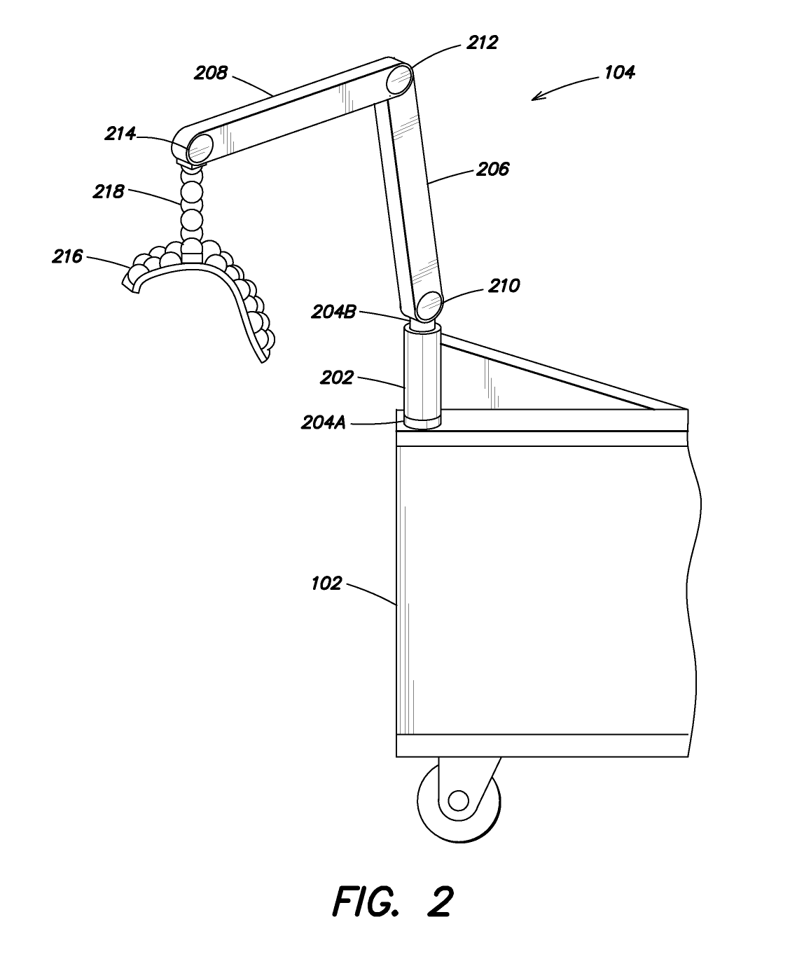

Referring now to FIG. 2, specifically, various embodiments of the coil support structure 104 can include a positioning arm that includes a base 202 configured to swivel at 204A and/or 204B. Either one or both of 204A-B can be constructed of swivel joints that allow the coil support structure to be rotated into and out of an imaging position. Base 202 can be connected to first 206 and second 208 support arm members. In some embodiments, the connections between the base 202, the first support arm member 206, and the second support arm member 208 can include articulating joints 210-212 configured to permit movement of the support arm. In some embodiments, the joints 210-212 are gimbal joints permitting movement in all directions, in other embodiments, the joints 210-212 can be hinge joints that permit movement in the plane of the support arm, and swivel joints can be used to rotate the support arm. In yet other embodiments, other types of joints can be used or combinations of any type of joints at 210-212.

According to another embodiment, a supine coil assembly 216 can be connect to a distal end of the second support arm member 208 at a joint 214. Joint 214 can be constructed of a hinge joint or a ball and socket joint that permit movement of the supine coil assembly 216. In some embodiments, the joint 214 can be connected to a hanger arm 218. The hanger arm can be configured to articulate to further position the supine coil array 216. In some embodiments, hanger arm 218 can include a plurality of ball and socket joints that can manipulated in any direction enabling more precise positioning of the supine coil array. Other coil support structures can also be used in various embodiments. For example, U.S. Pat. No. 7,731,662 discloses an example of movable support arm that can be used in conjunction with the embodiments disclosed herein as a coil support structure. In one example, the moveable support arm disclosed can be connected to a hanger arm (e.g., 216) of a supine coil. According to some embodiments, the coil support structure and/or hanger arm 216 are configured to permit movement of support structure and coil array, so that, for example, a patient can easily be positioned on, for example, patient transport 102.

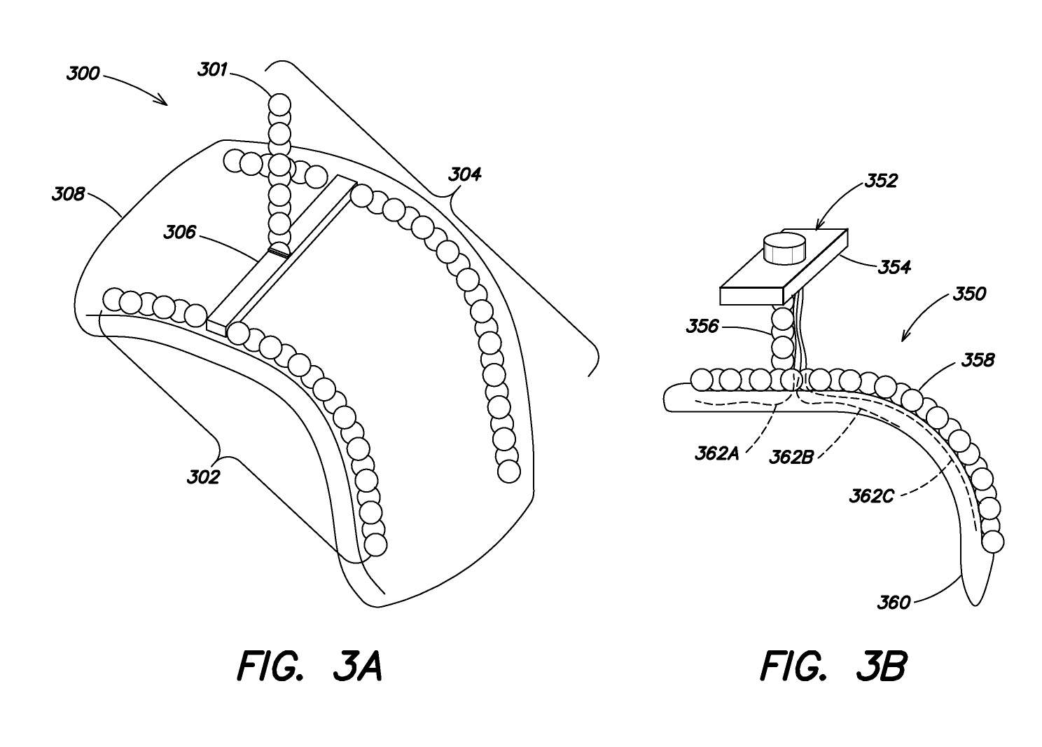

Referring to FIG. 3B, an example coil assembly 350 includes hanger assembly 352 which can be connected to or at a coil support structure (e.g., 104). Hanger assembly 352 can include a cross piece 354, which can also permit connections to a variety of support structures configured to hold a supine coil above a patient for MR imaging. Hanger assembly 352 can be connected to hanger arm 356. Hanger arm 356 can be constructed of a plurality of joints which enable articulation of the hanger arm 356 in any direction. Hanger arm 356 is further configured to hold a position into which it is articulated. In some examples, the plurality of joints are each pressure fit to hold whatever configuration they are moved into. In one example, the plurality of joints can be pressure fit gimbaled joints, that allow positioning of the coil assembly 350 by application of pressure on hanger arm 356, and once pressure is release hanger arm 356 holds its new position.

In another example, once a connected support structure is positioned over a patient, the hanger arm 356 enables finer positioning of a supine coil array over, for example, a patient's breast. Appropriate spacing between the imaged tissue and the coil assembly can be facilitated by hanger arm 356. And further, adjustments to positioning can be made if, for example, visual observation of the patient indicates that assembly 350 contacts the patient's breast or adjacent portions of the patient's body.

In some embodiments, the hanger arm 356 can be connected to an articulating structure of the coil assembly 350. The articulating structure can be constructed of a plurality of articulating members including, for example, a first articulating member 358. The plurality of articulating members can be connected to sheet of flexible material that makes of at least a portion of a base 360 of the coil assembly 350. The base 360 of the coil assembly can include the imaging circuitry, for example, wires 362A-C that can be configured to carry data and/or power between the coil assembly and a MR scanner and/or the MR scanner controller.

Shown in FIG. 3A is another embodiment of a supine coil assembly 300 including a hanger arm 301 which can be connect to a supine coil support structure. The supine coil assembly includes a first and a second articulating member 302-304 that are configured to, for example, shape the supine coil so as to conform to the contours of the patient's breast and adjacent portions of the patient's body. As discussed above, the articulating members can be constructed of a plurality of joints that are moveable and configured to retain their position once placed. Thus, the supine coil can be configured to follow the contours of the patient's anatomy using the articulating members. In some embodiments, the supine coil is positioned to preserve an air gap between the coil and the patient's skin, while following the contours of the patient's body as closely as possible.

The first and second articulating members 302-304 can be connected by a cross support beam 306. In some embodiments, the cross support can be a rigid support member. In other embodiments, the cross support can also be an articulating member (e.g., Loc-Line system, Modular Hose, assembly of a plurality of joints, etc.) that permits further shaping and/or configuration of the coil assembly 300.

In some embodiments, the shaping/or and positioning of the coil assembly ensures optimal coupling to the breast tissue, while allowing the breast to remain in its native supine configuration. According to one aspect, enabling the patient's breast to remain in its native supine configuration improves correspondence between supine images and visual observation of the imaged tissue that is expected to be in the same position in subsequent surgical or interventional procedures

In further embodiments, additional articulating members can be integrated into the coil assembly 300, and for example, connected to a base 308 of the assembly 300. In some examples, each articulating member can include flexible ball-and-socket portions, which are configured to permit reshaping of each of the first and second articulating members and corresponding changes in the shape of the base 308. According to some embodiments, the base 308 can be constructed and arranged of a flexible sheet of material. In one example, base 308 is constructed of Teflon which supports the elements of an RF coil.

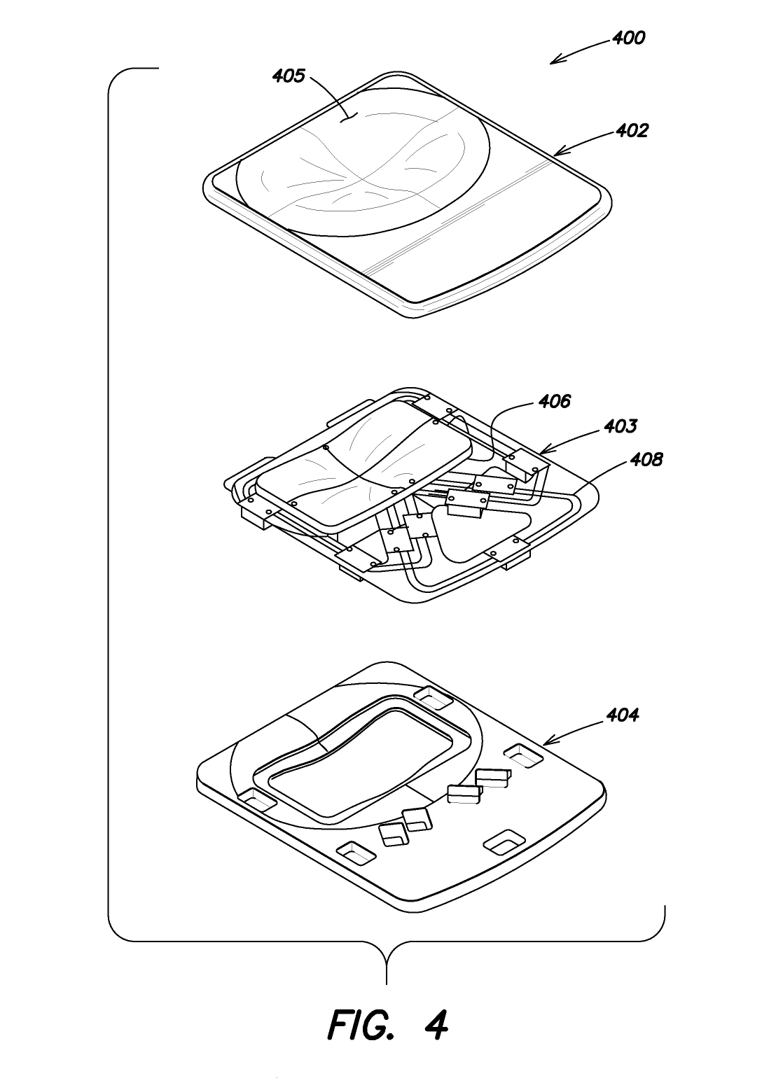

According to one embodiment, the articulating members are connected to a Teflon base 308 which supports an RF coil. In some embodiments the RF coil is integrated into the Teflon base, and in others can be incorporated between molded Teflon layers (e.g., FIG. 4, discussed in greater detail below). In further embodiments, the RF coil circuitry can be printed onto a Teflon base. In still other embodiments, the base 308 and an RF coil can be molded around the articulating members.

Shown in FIG. 4 is an exploded view of example base 400 of a supine coil assembly according to one embodiment. Base 400 can be constructed of a flexible material, including, for example, Teflon. The base 400 can be fixed to an articulating structure, for example, as shown in FIGS. 3A-B. According to one embodiment, base 400 includes a plurality of layers 402, 403, and 404. The upper layer 402 when assembled is positioned towards a patient to be imaged. A cup region can be defined in the base 400, and more particularly, a cup region 405 can be defined to include a shallow depression that permits conformity to a naturally positioned breast. Cup region 405 can also be constructed of a flexible material, including Teflon. The cup region 405 can be re-shaped or reconfigured based on articulation of articulating members (not shown) in a coil assembly. By reshaping the cup region, a supine coil assembly can be readily configured to accommodate various geometries of breasts, and further various geometries of adjacent body areas can be accommodated by further configuration of the articulating members.

In some embodiments, an intermediate layer 403 is disposed underneath the upper layer 402. The intermediate layer 403 can include RF coil arrays, e.g., 406 used for imaging. An RF antenna or array of RF antennas 408 can be attached to the RF coil array 406. The antenna or array of RF antennas 408 can be coupled between a lower layer 404 or base layer and upper layer 402 or upper housing. The RF antenna 408, therefore, can be positioned to be directed toward the chest wall of the patient when the patient is resting in a supine position and the coil is placed adjacent to the patient's breast. For example, the patient shown in FIG. 1 is resting in a supine position and the coil assembly may be positioned directly above the patient's breast and adjacent body areas.

In further embodiments, coils are arranged to provide imaging coverage of breast tissue, and may also be arranged to provide additional coverage of the medial steinum and axilla or other body areas adjacent to the breast by manipulating the shape of the supine coil.

Referring to FIGS. 3A-3B, to adjust the position of the RF coil 406 and/or RF antennas 408 relative to the position of a breast of the patient position in a supine position, the first and second articulating members 302-304 are adjusted to conform to the contour of the patient's breast resulting in the repositioning of the RF coil and/or RF antennas 408 disposed in the base of the coil assembly (e.g., 400). The articulating members 302-304 are adjustable to configure, for example, the base 300 along the contour of the patient's breast and associated clinically relevant areas around the breast for imaging.

According to one embodiment, a supine coil array was constructed from a unilateral four-element receive coil. In one example, the receive coil elements are constructed to have a size of 26×23 cm. In the example elements, the four-element receive coil was constructed an arranged of 6.35 mm wide copper tape, although in other embodiments, different sizes and different materials can be used for receive coil circuitry. Further, in some embodiments, coupling between coil loops was minimized by overlapping neighboring loops. Further embodiments also include architectures to decouple capacitors, and still further embodiments also include low-impedance pre-amplifiers, which features can be implemented to improve signal-to-noise (“SNR”) ratios for image capture.

According to one embodiment, the supine coil array may be used in practice as follows. A patient may be supinely positioned upon an examination bed proximate to the supine coil. The supine coil is lowered proximate to the breast of the patient. The medical professional then manipulates the supine coil until it is proximate to, but preferably not in contact, with the patients breast. A flattened portion of the coil may be extending over the medial tissue of the patient, i.e., over the rib cage, and also flattened down over the axilla tissue, i.e., under the arm and down the rib cage. The patient may then be positioned inside an MRI scanner for image capture. Following image capture, the patient may be transported, in the same position, for further treatment. With such an arrangement, because the patient may remain in the same position for imaging and treatment, the speed and accuracy of any treatment is greatly increased.

Accordingly, a supine coil which is capable of being contoured to provide closely conform to the breast anatomy has been shown and described. Provision of a contoured supine coil of the present invention prevents distortion of the breast and enables imaging of diagnostic quality and high SNR

The ability to perform supine imaging, using the coil of the present invention, mimics the breast configuration of most clinical procedures more closely than that of prone breast MRI. Providing improved correspondence between imaging positioning and procedure positioning can substantially improve post-imaging registration accuracy, according to further embodiments.

It is to be appreciated that embodiments of the methods and apparatuses discussed herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiments.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. An imaging array for imaging tissue of a patient positioned in a supine position, the imaging array comprising:
a flexible mounting element, wherein the flexible mounting element includes at least one articulating member configured to flex into position responsive to pressure and to hold a flexed position upon release of pressure and a curved portion, sized and dimensioned for positioning adjacent to a naturally positioned breast in response to articulation of the at least one articulating member of the flexible mounting element; and
an RF coil array disposed on the flexible mounting element.

2. The imaging coil array of claim 1, further comprising a positioning assembly configured to position the flexible mounting element adjacent to the supine positioned patient.

3. The imaging coil array of claim 2, wherein the positioning assembly is further configured to position the flexible mounting element adjacent to the supine positioned patient without deforming the naturally positioned breast while the supine positioned patient is breathing.

4. The imaging coil array of claim 2, wherein the positioning assembly comprises components selected from a group including articulating components, telescoping components and flexible components.

5. The imaging coil array of claim 1, wherein the at least the at least one articulating member is constructed and arranged of a plurality of articulating connectors.

6. The imaging coil array of claim 5, wherein each of the plurality of articulating connectors are configured to hold a fixed position until a threshold pressure is applied, and wherein the plurality of articulating connectors are configured to permit movement when the threshold pressure is exceeded.

7. The imaging coil array of claim 5, wherein the plurality of articulating connectors include pressure fit joints.

8. The imaging coil array of claim 3, wherein the flexible mounting element includes a configuration having a generally flat flexible portion adjacent to the curved portion.

9. The imaging coil array of claim 8, wherein the generally flat flexible portion the flexible mounting element is configured to conform to contours of a body of the supine positioned patient adjacent the breast and positioned to permit breathing without touching the body of the supine positioned patient.

10. The imaging coil array of claim 3, wherein the curved portion is configured to conform to a contour of a plurality of breast sizes responsive to articulation of the at least the at least one articulating member of the flexible mounting element.

11. The imaging coil array of claim 10, wherein the curved portion is positionable for imaging the naturally positioned breast during breathing without touching any imaged tissue.

12. A method of obtaining an Magnetic Resonance Image (MRI) of an object, including the steps of:
positioning an RF coil for use in MRI imaging above the breast of a patient that is in a supine imaging position on a transportable patient bed;
contouring the RF coil to conform to the shape of the patient's breast;
moving the transportable patient bed into an MRI scanner and obtaining an MRI image; and
while the patient remains in the supine imaging position, providing medical treatment to the patient, the medical treatment guided by at least some information in the MRI image.

13. An imaging array for imaging tissue of a patient positioned in a supine position, the imaging array comprising:
a flexible mounting element that includes at least a first articulating member having a first position and that is configured to flex into a flexed position responsive to pressure and hold the flexed position upon release of pressure;
an RF coil array disposed on the flexible mounting element; and
and a cup-shaped region formed on the flexible mounting element, wherein the cup-shaped region is configured to be sized and dimensioned responsive to articulation of the at least one articulating member of the flexible mounting element.

14. The imaging coil array of claim 13, further comprising a positioning assembly configured to position the flexible mounting element adjacent to the supine positioned patient.

15. The imaging coil array of claim 13, wherein the at least the first articulating member is constructed and arranged of a plurality of articulating connectors.

16. The imaging coil array of claim 15, wherein each of the plurality of articulating connectors are configured to hold the first position until a threshold pressure is applied, and wherein the plurality of articulating members are configured to permit movement into the flexed position when the threshold pressure is exceeded.

17. The imaging coil array of claim 7, wherein the plurality of articulating connectors include components selected from a group including articulating components, telescoping components and flexible components.

18. The method of claim 12, further comprising an act of maintaining a spacing between the RF coil and the breast of the patient.

19. The method of claim 12, wherein the contouring of the RF coil includes applying a threshold pressure to the RF coil so as to conform the shape of the RF coil to the patient's breast.

20. The method of claim 12, further comprising an act of contouring the RF coil to conform to a shape of another patient's breast.
